(19) United States
(12) Patent Application Publication
(10) Pub. No.: US 2023/0140522 A1
(43) Pub. Date: May 4, 2023

(54) COMPOSITION FOR AMELIORATING OR PREVENTING DECREASES IN BONE STRENGTH

(71) Applicant: AJINOMOTO CO., INC., TOKYO (JP)

(72) Inventors: Yuko Susa, Kanagawa (JP); Reiko Ichikawa, Kanagawa (JP); Yoshiko Inoue, Kanagawa (JP); Chie Tanaka, Kanagawa (JP); Katsuya Suzuki, Kanagawa (JP)

(73) Assignee: AJINOMOTO CO., INC., TOKYO (JP)

(21) Appl. No.: 17/956,357

(22) Filed: Sep. 29, 2022

Related U.S. Application Data
(63) Continuation of application No. PCT/JP2021/013624, filed on Mar. 30, 2021.

(30) Foreign Application Priority Data
Mar. 30, 2020 (JP) 2020-060915

(51) Int. Cl. A61K 31/198; A61P 19/10

(52) U.S. Cl. CPC A61K 31/198; A61P 19/10

(57) ABSTRACT

An aspect is to provide a composition effective for preventing or improving bone strength decrease. A composition for preventing or improving bone strength decrease, that is, bone strength decreases due to bone density decrease, bone quality decrease, bone metabolism decrease, and the like, containing ornithine as an active ingredient.

### STRENGTH

This application is a Continuation of, and claims priority under 35 U.S.C. § 120 to, International Application No. PCT/JP2021/013624, filed Mar. 30, 2021, and claims priority therethrough under 35 U.S.C. § 119 to Japanese Patent Application No. 2020-060915, filed Mar. 30, 2020, the entireties of which, as well as all citations cited herein, are incorporated by reference herein.

### BACKGROUND

The present invention relates to a composition for preventing or improving bone strength decrease.

### Background Art

Osteoporosis is a disease characterized by decreased bone strength and increased risk of bone fracture, and this characterization is generally accepted.

Bone strength is defined by two factors: bone density and bone quality. Bone density is determined by the amount of minerals such as calcium and the like that make up the bone (bone mass), and bone quality is defined by the material property which is the quality of the raw material of the bone, and the structural property (microstructure) built up from the material.

Since bone strength is defined by bone density and bone quality, if either of them decreases, the decrease in bone strengths and the risk of bone fracture increases. As one of the factors that decrease bone strength, it is known that accelerated bone resorption and decreased bone formation occur due to estrogen deficiency (menopause) and aging, which in turn leads to decreased bone density and deterioration of bone quality (Non Patent Literature 6).

In order to maintain the bone strength in a healthy state, a balance between bone formation by osteoblasts and bone resorption by osteoclasts (balance of bone metabolism) is important. It is known that one of the causes of osteoporosis is an imbalance in bone metabolism (bone resorption becomes stronger than bone formation) due to aging or menopause.

In addition, it is known that BMP-2 (Bone morphogenetic protein-2) signal, which is a differentiation factor into osteoblasts, decreases in elderly people, osteoporosis patients, and postmenopausal women (Non Patent Literatures 7 to 9).

It has been reported that ornithine is effective in improving liver function (Non Patent Literatures 1 and 2), reducing mental stress (Non Patent Literature 3), and recovering from fatigue (Non Patent Literatures 4 and 5).

However, the effect of ornithine on bone strength has not been reported to date.

### Citation List

NPL 1: Hishida. Food Style 21 16(11) 87-9, 2012

NPL 2:

Tamai et al., Amino Acids, 45 (6), 1343-51, 2013

NPL 3:

Miyake et al., Nutrition Journal 13, 53, 2014

NPL 4:

Sugino et al., Nutrition Research, 28 (11) 738-743, 2008

NPL 5:

Kokubo et al., BioPsychoSocial Medicine, 7, 6, 2013

NPL 6:

Guideline for prevention and treatment of osteoporosis 2015, Life Science Publishing Co., Ltd.

NPL 7:

Mehrunnisa M. Raje et al., Molecular Biology Reports (2019) 46: 1667-1674

NPL 8:

Fleet et al., Endocrinology, 137 (11), 4605, 1996

NPL 9:

Zhang et al., Exp Ther Med. 18(5), 3659-3666, 2019

### SUMMARY

An aspect of the present invention is to provide a composition effective for preventing or improving bone strength decrease.

It is shown in the Experimental Examples 1 to 4 described herein that ornithine has an osteoblast differentiation promoting action, that the osteoblast differentiation promoting action of ornithine is exhibited even under low BMP-2 concentrations, and further that ornithine has an osteoclast differentiation suppressive action.

It is described herein that ornithine can balance bone metabolism by suppressing decrease in bone formation and promotion of bone resorption (see the above-mentioned Non Patent Literature 6), which are known factors that decrease bone strength, and can be effectively used to prevent or improve bone strength decrease.

In addition, it is described herein that ornithine can be effectively used to prevent or improve bone strength decrease also in elderly people, osteoporosis patients, and postmenopausal women with reduced BMP-2 signals.

It is described herein, as shown in the Experimental Examples 5 to 8, ornithine has a bone density decrease suppressive effect in vivo, also promotes the expression of osteocalcin in vivo, suppresses muscle atrophy that occurs simultaneously with osteoporosis, suppresses the expression of muscle atrophy marker genes MuRF1 and Atg3, suppresses increase in liver weight, which is an indicator of fatty liver induced by OVX (ovariectomy), and suppresses the expression of SREBP1c, MTP, and PPARγin the liver.

It is an aspect of the present invention to provide a composition for preventing or improving bone strength decrease, comprising ornithine as an active ingredient.

It is a further aspect of the present invention to provide the composition as described herein, wherein the bone strength decrease is caused by menopause, immobilization osteoporosis, disuse osteoporosis, and/or aging.

It is a further aspect of the present invention to provide the composition as described herein, wherein the bone strength decrease is caused by bone density decrease or bone quality decrease.

It is a further aspect of the present invention to provide the composition as described herein, wherein the bone quality decrease is caused by bone metabolism decrease.

It is a further aspect of the present invention to provide the composition as described herein, wherein the composition is an osteoblast differentiation promoter and/or an osteoclast differentiation inhibitor.

It is a further aspect of the present invention to provide the composition as described herein, wherein the composition is able to prevent or improve musculoskeletal function decrease.

It is a further aspect of the present invention to provide the composition as described herein, wherein the composition is for ingestion by a subject with a decreased BMP-2 signal.

It is a further aspect of the present invention to provide the composition as described herein, wherein the subject with a decreased BMP-2 signal is a postmenopausal woman, an osteoporosis patient, or an elderly person.

It is a further aspect of the present invention to provide a composition for inducing osteocalcin expression, comprising ornithine as an active ingredient.

It is a further aspect of the present invention to provide a composition for preventing or improving changes in physical condition due to changes in postmenopausal hormone balance, comprising ornithine as an active ingredient.

It is a further aspect of the present invention to provide the composition as described above, wherein the change in the physical condition is caused by a bone strength decrease and/or a liver weight increase.

It is a further aspect of the present invention to provide the composition as described above, wherein the composition is a food.

It is a further aspect of the present invention to provide the composition as described above, wherein the composition is indicated for a function resulting from osteocalcin expression induction.

It is a further aspect of the present invention to provide the composition as described above], wherein the function is selected from the group consisting of “prevention of osteoporosis”, “strengthening bone”, “promotion of regeneration of bone”, “bone building capacity”, “suppression of bone fracture”, and “acceleration of recovery from bone fracture”.

It is a further aspect of the present invention to provide a composition for suppressing the expression of MuRF1 and/or Atg3, comprising ornithine as an active ingredient.

It is a further aspect of the present invention to provide the composition as described above], wherein the composition is indicated for a function resulting from suppressing the expression of MuRF1 and/or Atg3.

It is a further aspect of the present invention to provide the composition as described above, wherein the function is selected from the group consisting of “maintaining body in movable state for a long time”, “maintaining walking function”, “maintaining exercise function”, and “being able to walk for a long time”.

It is a further aspect of the present invention to provide a composition for suppressing the expression of SREBP1c and/or MTP and/or PPARγ, comprising ornithine as an active ingredient.

It is a further aspect of the present invention to provide the composition as described above, wherein the composition is indicated for a function resulting from suppressing the expression of SREBP1c and/or MTP and/or PPARγ.

It is a further aspect of the present invention to provide a method for preventing or improving a bone strength decrease in a subject in need of the prevention or improvement of bone strength decrease, comprising administering a composition comprising an effective amount of ornithine to the subject.

It is a further aspect of the present invention to provide the method as described above, wherein the bone strength decrease is caused by menopause, immobilization osteoporosis, disuse osteoporosis, and/or aging.

It is a further aspect of the present invention to provide the method as described above, wherein the bone strength decrease is caused by bone density decrease or bone quality decrease.

It is a further aspect of the present invention to provide the method as described above, wherein the bone quality decrease is caused by bone metabolism decrease.

It is a further aspect of the present invention to provide the method as described above, wherein the prevention or improvement of bone strength decrease is achieved by the promotion of osteoblast differentiation and/or suppression of osteoclast differentiation.

It is a further aspect of the present invention to provide the method as described above, wherein the administration subject is in further need of the prevention or improvement of musculoskeletal function decrease.

It is a further aspect of the present invention to provide the method as described above, wherein the administration subject is a subject with a decreased BMP-2 signal.

It is a further aspect of the present invention to provide the method as described above, wherein the subject with a decreased BMP-2 signal is a postmenopausal woman, an osteoporosis patient, or an elderly person.

It is a further aspect of the present invention to provide a method for inducing osteocalcin expression in a subject in need of the induction of osteocalcin expression, comprising administering a composition comprising an effective amount of ornithine to the subject.

It is a further aspect of the present invention to provide a method for preventing or improving changes in physical condition due to changes in postmenopausal hormone balance in a subject in need of the prevention or improvement of changes in physical condition due to changes in postmenopausal hormone balance, comprising administering a composition comprising an effective amount of ornithine to the subject.

It is a further aspect of the present invention to provide the method as described above, wherein the change in the physical condition is caused by bone strength decrease and/or liver weight increase.

It is a further aspect of the present invention to provide the method as described above, wherein the composition is orally administered.

It is a further aspect of the present invention to provide a method for suppressing the expression of MuRF1 and/or Atg3 in a subject in need of the suppression of the expression of MuRF1 and/or Atg3, comprising administering a composition comprising an effective amount of ornithine to the subject.

It is a further aspect of the present invention to provide a method for suppressing the expression of SREBP1c and/or MTP and/or PPARγin a subject in need of the suppression of the expression of SREBP1c and/or MTP and/or PPARγ, comprising administering a composition comprising an effective amount of ornithine to the subject.

The composition containing ornithine as described herein can be effectively used for the prevention or improvement of bone strength decrease, based on the osteoblast differentiation promoting action, and/or osteoclast differentiation suppressive action of ornithine. Particularly, since ornithine shows an osteoblast differentiation promoting action even at low BMP-2 concentrations, the composition containing ornithine is advantageous in that it can be used effectively for the prevention or improvement of bone strength decrease even in elderly people, osteoporosis patients, and postmenopausal women, showing decreased BMP-2 signal.

The composition containing ornithine can be effectively used for the prevention or improvement of musculoskeletal function decrease such as disuse muscle atrophy and the like in subjects with decreased bone strength such as osteoporosis and the like.

The composition containing ornithine can be effectively used for the prevention or improvement of changes in physical condition (poor physical condition) due to changes in postmenopausal hormone balance, particularly, changes in physical condition (poor physical condition) caused by bone strength decrease and/or liver weight increase.

### BRIEF DESCRIPTION OF DRAWINGS

FIG. 1 shows the results of Experimental Example 1.

FIG. 2 shows the results of Experimental Example 2.

FIG. 3 shows the results of Experimental Example 3. In FIG. 3, abbreviation Orn indicates ornithine.

FIG. 4 shows the results of Experimental Example 4.

FIG. 5 shows the experiment schedule for OVX (ornithine administration) group in Experimental Example 5.

FIG. 6 shows the results of Experimental Example 5.

FIG. 7 shows the experiment schedule for OVX (ornithine 1% blended feed administration with cast fixation) group and OVX (ornithine 3% blended feed administration with cast fixation) group in Experimental Example 6.

FIG. 8 shows the results of Experimental Example 6.

FIG. 9 shows the results of Experimental Example 7.

FIG. 10 shows the results (osteocalcin expression level) of Example 8.

FIG. 11 shows the results (MuRF1 and Atg3 expression level) of Example 8.

FIGS. 12-1 shows the results (SREBP1c and MTP expression levels) of Example 8.

FIGS. 12-2 shows the results (PPARγ expression level) of Example 8.

### DETAILED DESCRIPTION OF THE EXEMPLARY EMBODIMENTS

The composition for preventing or improving bone strength decrease contains ornithine as an active ingredient.

The “bone strength decrease” includes bone density (bone mass) decrease and bone quality decrease (e.g., bone metabolism decrease).

The “improvement of bone strength decrease” refers to bringing the decreased bone strength back to a healthy state or close to a healthy state, and the “prevention of bone strength decrease” refers to maintaining bone strength in a healthy state, or preventing further deterioration of the decreased bone strength.

The factors causing bone strength decrease are not particularly limited and include, for example, menopause, immobilization osteoporosis, disuse osteoporosis, aging, malnutrition (e.g., malnutrition in young women), young age (e.g., growth phase), bone strength decrease due to lack of exercise.

The composition is particularly useful for bone strength decrease due to menopause, immobilization osteoporosis, disuse osteoporosis, and aging.

Particularly, the composition is useful in that it can also be used for subjects with low BMP-2 signal (e.g., postmenopausal women, osteoporosis patients, elderly people).

The composition can be used as an osteoblast differentiation promoter and/or an osteoclast differentiation inhibitor, since ornithine as the active ingredient shows an osteoblast differentiation promoting action, and an osteoclast differentiation suppressive action, as shown in the Experimental Examples herein.

The composition can be formulated with indication of a function.

Examples of the indication of a function include “prevention of osteoporosis”, “strengthening bone”, “promotion of regeneration of bone”, “bone building capacity”, “suppression of bone fracture”, and “acceleration of recovery from bone fracture”.

The composition for preventing or improving bone strength decrease can be further used for preventing or improving musculoskeletal function decrease.

The “musculoskeletal function” includes a function of moving limbs and body and, for example, walking function can be mentioned.

The “musculoskeletal function decrease” includes musculoskeletal function decrease due to disuse muscle atrophy.

The “improvement of musculoskeletal function decrease” refers to bringing the decreased musculoskeletal function back to a healthy state or close to a healthy state, and the “prevention of musculoskeletal function decrease” refers to maintaining musculoskeletal function in a healthy state, or preventing further deterioration of the decreased musculoskeletal function.

The composition is useful in that it can be expected to achieve both effects of prevention or improvement of bone strength decrease and prevention or improvement of musculoskeletal function decrease, by administration to subjects who have simultaneously developed bone strength decrease, such as osteoporosis and the like, and musculoskeletal function decrease, such as disuse muscle atrophy and the like.

The composition can be formulated with indication of a function.

Examples of the indication of a function include “maintaining body in movable state for a long time”, “maintaining walking function”, “maintaining exercise function”, and “being able to walk for a long time”.

While ornithine as the active ingredient may be in any of L-form, D-form, and DL-form, L-form is a particular example.

Ornithine may be in the form of a salt. As the salt, a salt acceptable as a food or medicament can be mentioned. Examples thereof include alkali metal salts such as sodium salt, potassium salt, and the like; alkaline earth metal salts such as calcium salt, magnesium salt, barium salt, and the like; aluminum salt; salts with organic bases such as ethylenediamine, propylenediamine, ethanolamine, monoalkyl ethanolamine, dialkyl ethanolamine, diethanolamine, triethanolamine, and the like; salts with inorganic acids such as hydrochloric acid, hydrobromic acid, nitric acid, sulfuric acid, phosphoric acid, and the like; and salts with organic acids such as formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid and the like.

The amount of ornithine present in the composition can be, for example, 0.01 - 100 wt%, 0.1 - 95 wt%, 1 - 90 wt%, or 5 -80 wt%.

While the composition can be used as a food, a medicament, and the like, use in a food is a particular example.

Food can be a concept that broadly includes foods that can be ingested orally (excluding pharmaceutical products), and includes not only so-called “food” but also beverages, health supplement, food with health claims (e.g., food for specified health uses, food with functional claims), supplement, and the like.

The form of the composition is not particularly questioned and may be, for example, powder, granule (including fine granule), tablet, hard capsule, soft capsule, liquid (e.g., solution, suspension, emulsion), drink, jelly, pudding, yogurt, candy, chewing gum, or the like. These can be produced by a known method. For example, the composition is mixed with carriers (e.g., excipient, binder, disintegrant, lubricant, solvent) and powder, granule, tablet, capsule, liquid, and the like can be produced by a method known in the field of food preparation or pharmaceutical preparation. In addition, the composition can also be produced by adding and mixing to and with food and drink (e.g., water, soft drink).

The ingestion amount (dose) of ornithine in the composition can be, for example, 1 mg to 24 g, 50 mg to 24 g, 100 mg to 12 g, or 200 mg to 4.8 g, per day for an adult (body weight 60 kg).

The composition can be safely ingested by (administered to), human, animals other than human (e.g., mammals and birds such as domestic animals, poultry, experiment animals). The form of ingestion (administration) for animals other than human may be addition to a feed.

In one embodiment, a composition for inducing osteocalcin expression is described, containing ornithine as an active ingredient.

In the composition for inducing osteocalcin expression, the definition of ornithine, the amount of ornithine, availability for food, medicament, and the like, form, ingestion amount, subject of ingestion and administration, and the like are the same as the definition of ornithine, the amount of ornithine, availability for food, medicament, and the like, form, ingestion amount, subject of ingestion and administration, and the like in the above-mentioned composition for preventing or improving bone strength decrease.

The osteocalcin expression induction can be confirmed, for example, by a method according to Experimental Example 8.

The composition can be expected to achieve effects such as “prevention of osteoporosis”, “strengthening bone”, “promotion of regeneration of bone”, “bone building capacity”, “suppression of bone fracture”, “acceleration of recovery from bone fracture”, and the like since ornithine as the active ingredient has an osteocalcin expression inducing action.

The composition for inducing osteocalcin expression can be formulated as a composition with indication of a function resulting from induction of osteocalcin expression.

Examples of the indication of a function include “prevention of osteoporosis”, “strengthening bone”, “promotion of regeneration of bone”, “bone building capacity”, “suppression of bone fracture”, and “acceleration of recovery from bone fracture”.

In one embodiment, a composition for suppressing the expression of MuRF1 and/or Atg3, containing ornithine as an active ingredient is described.

In the composition for suppressing the expression of MuRF1 and/or Atg3, the definition of ornithine, the amount of ornithine, availability for food, medicament, and the like, form, ingestion amount, subject of ingestion and administration, and the like are the same as the definition of ornithine, the content of ornithine, availability for food, medicament, and the like, form, ingestion amount, subject of ingestion and administration, and the like in the above-mentioned composition for preventing or improving bone strength decrease.

The suppression of the expression of MuRF1 and Atg3 can be confirmed, for example, by a method according to Experimental Example 8.

The composition can be expected to achieve effects such as “maintaining body in movable state for a long time”, “maintaining walking function”, “maintaining exercise function”, “being able to walk for a long time” and the like since ornithine as the active ingredient has a suppressive action on the expression of MuRF1 and Atg3.

The composition for suppressing the expression of MuRF1 and/or Atg3 can be formulated as a composition with indication of a function resulting from suppressing the expression of MuRF1 and/or Atg3.

Examples of the indication of a function include “maintaining body in movable state for a long time”, “maintaining walking function”, “maintaining exercise function”, and “being able to walk for a long time”.

In one embodiment, a composition for suppressing the expression of SREBP1c and/or MTP and/or PPARγ, containing ornithine as an active ingredient is described.

In the composition for suppressing the expression of SREBP1c and/or MTP and/or PPARγ, the definition of ornithine, the amount of ornithine, availability for food, medicament, and the like, form, ingestion amount, subject of ingestion and administration, and the like are the same as the definition of ornithine, the amount of ornithine, availability for food, medicament, and the like, form, ingestion amount, subject of ingestion and administration, and the like in the above-mentioned composition for preventing or improving bone strength decrease.

The suppression of the expression of SREBP1c, MTP, and PPARγcan be confirmed, for example, by a method according to Experimental Example 8.

The composition can be expected to achieve effects such as improvement of fatty liver and hyperlipidemia, and the like since ornithine as the active ingredient has a suppressive action on the expression of SREBP1c, MTP, and PPARγ.

The composition for suppressing the expression of SREBP1c and/or MTP and/or PPARγcan be formulated with indication of a function resulting from suppressing the expression of SREBP1c and/or MTP and/or PPARγ.

In one embodiment, a composition for preventing or improving changes in physical condition due to changes in postmenopausal hormone balance, containing ornithine as an active ingredient is described.

As the changes in physical condition due to changes in postmenopausal hormone balance, changes in physical condition caused by decreased bone strength and/or increased liver weight can be mentioned. Specific examples of the changes in physical condition include bone fracture, decreased exercise, muscle atrophy associated with decreased exercise and decreased bone density, fatty liver, and hyperlipidemia.

In the composition for preventing or improving changes in physical condition due to changes in postmenopausal hormone balance, the definition of ornithine, the amount of ornithine, availability for food, medicament, and the like, form, ingestion amount, subject of ingestion and administration, and the like are the same as the definition of ornithine, the amount of ornithine, availability for food, medicament, and the like, form, ingestion amount, subject of ingestion and administration, and the like in the above-mentioned composition for preventing or improving bone strength decrease.

The composition can be formulated with indication of a function.

Examples of the indication of a function include “supporting changes in women’s physical condition” and “improving poor physical condition in postmenopausal women”.

### EXAMPLES

The present invention is explained in more detail in the following by referring to Experimental Examples. The present invention is not limited to these.

### Experimental Example 1: Alkaline Phosphatase (Osteoblast Differentiation Marker) Activity Measurement Test After Ornithine Addition, by Using Osteoprogenitor Cell MC3T3-E1

Using the following materials and methods, osteoprogenitor cells were cultured in ornithine-added or ornithine-free medium, the alkaline phosphatase activity in the cultured cells was measured, and the osteoblast differentiation-promoting action of ornithine was examined.

### Materials

(1) Cell
   MC3T3-E(RIKEN Cell Bank, RCB1126)
(2) Medium
   α-MEM medium, 10% FBS, antibiotic added
(3) Test reagent, etc.
   α-MEM medium (phenol red free) (Invitrogen, Cat. No. 41061-029)
Penicillin-streptomycin solution (nacalai tesque, Cat. No. 26253-84)
0.25% trypsin-EDTA solution (nacalai tesque, Cat. No. 32777-44)
Dulbecco’s PBS (Nissui Pharmaceutical Co., Ltd., Code No. 05913)
viable cell count measurement reagent SF (nacalai tesque, Cat. No. 07553-44)
BMP-2, Human Recombinant (R&D Systems, Cat. No. 355-BEC-010)
Micro BCA Protein Assay Reagent Kit (PIERCE, Cat. No. 23235)
Cell-LyEX1 (Wako, Cat. No. 300-34761)
LabAssay ALP (Wako, Cat. No. 291-58601)
Mouse Osteocalcin EIA kit (Biomedical Technologies, Cat. No. BT-470)
L-Ornithine Monohydrochloride (Nacalai, Cat. No. 25718-92)

### Method

### Cell Preculture

The cells were placed in a T-75 flask using a medium and cultured in a CO₂ incubator (5% CO₂, 37° C.). The medium was changed every other day, and the cells were collected when they reached 80% confluence and used for the test.

### Test

The cells were adjusted with medium to 1.2x10⁵ cells/0.2 mL/well and seeded in a 48-well plate. The next day, the medium was replaced with a medium supplemented with ornithine 0.2 mg/mL, 1.0 mg/mL or 5.0 mg/mL or additive-free medium, and the cells were cultured for 7 and 14 days. After culturing for 7 days, the cells were washed once with PBS for ALP activity measurement and then cryopreserved together with the plate. In addition, after culturing for 14 days, the culture supernatant was sampled for measurement of the amount of osteocalcin used in the below-mentioned Experimental Example 2, and cryopreserved. The test was performed with n=5 and the medium was changed every 3-4 days.

### Alkaline Phosphatase (ALP) Activity Measurement

The cells were washed with PBS and lysed with 60 µL/well of cell lysing agent (Cell-LyEX1 containing 2 mM PMSF). After stirring the plate at room temperature for 30 min, the supernatant was diluted 5-fold and the obtained solution was used as a sample for measurement. ALP activity in the cells was measured by LabAssay ALP. With this kit, ALP activity was measured from the amount of p-nitrophenol per unit amount of protein produced within a given period of time. The amount of protein in the solution was measured using Micro BCA Protein Assay Reagent Kit.

### Significant Difference Test

The test was a two-sided test with Student’s t-test, and P<0.05 (less than 5% of null hypothesis) or more was judged as significant difference. The data in the graph is shown in mean±standard error.

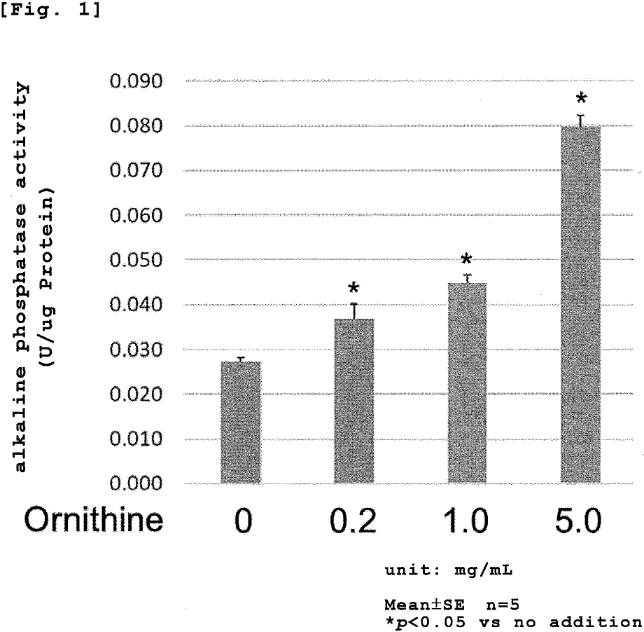

As shown in FIG. 1, alkaline phosphatase activity significantly increased by the addition of ornithine at 0.2 mg/mL, 1.0 mg/mL, and 5.0 mg/mL, as compared with no addition of ornithine.

The results show that ornithine promotes differentiation of osteoblasts.

### Experimental Example 2: Osteocalcin (Osteoblast Differentiation Marker) Production Amount Measurement Test After Ornithine Addition, by Using Osteoprogenitor Cell MC3T3-E1

Using the “culture supernatant for measurement of the amount of osteocalcin” sampled in “method (2) test” of the above-mentioned Experimental Example 1, the osteocalcin (osteoblast differentiation marker) production amount was measured by the following method, and the osteoblast differentiation promoting action of ornithine was investigated.

### Method

### Osteocalcin Amount Measurement

For the measurement of the amount of osteocalcin in the culture supernatant, the supernatant was diluted 10-fold and the measurement was performed using Mouse Osteocalcin EIA kit.

### Significant Difference Test

The test was a two-sided test with Student’s t-test, and P<0.05 (less than 5% of null hypothesis) or more was judged as significant difference. The data in the graph is shown in mean±standard error.

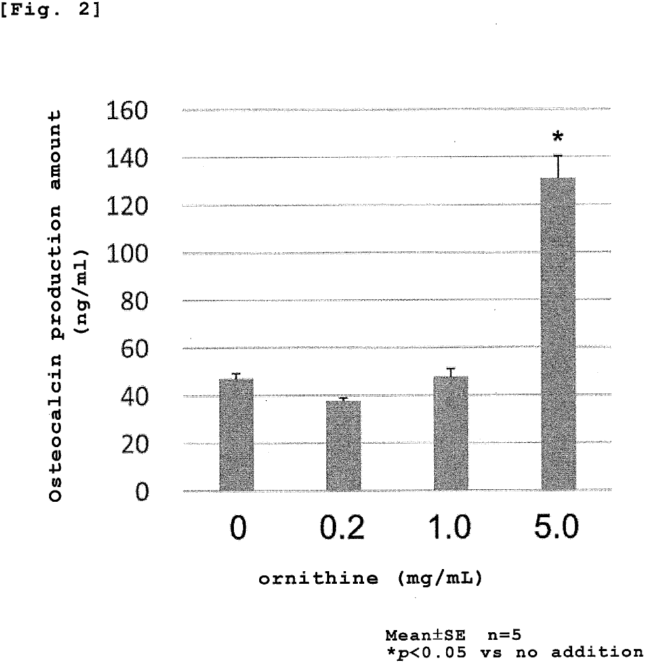

As shown in FIG. 2, osteocalcin production amount significantly increased by the addition of ornithine at 5.0 mg/mL, as compared with no addition of ornithine.

The results show that ornithine promotes differentiation of osteoblasts.

### Experimental Example 3 Alkaline Phosphatase (Osteoblast Differentiation Marker) Activity Measurement Test After Ornithine Addition, by Using Osteoprogenitor Cell MC3T3-E1 (Influence of BMP-2 (Differentiation Factor) Concentration)

Using the following methods, osteoprogenitor cells were cultured in an ornithine-added or ornithine-free medium with varying BMP-2 (differentiation factor) concentrations and, the alkaline phosphatase activity in the cultured cells was measured, and the influence of BMP-2 (differentiation factor) concentration on the osteoblast differentiation-promoting action of ornithine was examined.

### Method

### Cell Preculture

The cells were placed in a 10 cm petri dish using a medium and cultured in a CO₂ incubator (5% CO₂, 37° C.). The medium was changed every other day, and the cells were collected when they reached 80% confluence and used for the test.

### Test

The cells were adjusted with medium to 1.2x10⁵ cells/0.2 mL/well and seeded in a 48-well plate. The next day, the condition was changed to a medium containing BMP-2 at 50 ng/mL, 100 ng/mL, or 200 ng/mL and free of ornithine or containing 5 mg/mL ornithine, and the cells were cultured for 7 days. After 7 days, the cells were washed once with PBS, 50 µL of 0.1% Triton-X was added, and the mixture was further sonicated for 1 min to prepare a cell lysate. The test was performed with n=4 and the medium was changed every 3-4 days.

### Alkaline Phosphatase (ALP) Activity Measurement

ALP activity of the above-mentioned cell lysate was measured by LabAssay ALP. With this kit, ALP activity was measured from the amount of p-nitrophenol per unit amount of protein produced within a given period of time. The amount of protein in the solution was measured using Micro BCA Protein Assay Reagent Kit.

### Significant Difference Test

The test was a two-sided test with Student’s t-test, and P<0.05 (less than 5% of null hypothesis) or more was judged as significant difference. The data in the graph is shown in mean±standard error.

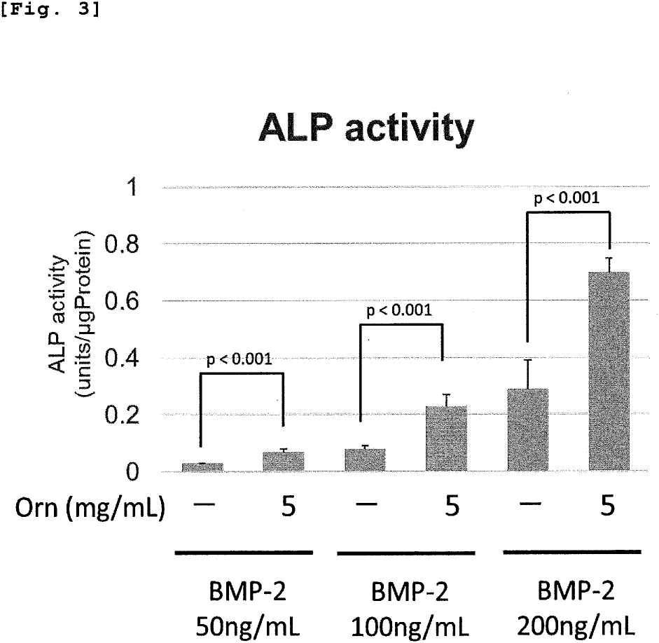

As shown in FIG. 3, alkaline phosphatase activity was significantly increased by BMP-2 concentrations of 50 ng/mL, 100 ng/mL, and 200 ng/mL, and ornithine 5 mg/mL addition, as compared with ornithine no addition, irrespective of the BMP-2 addition concentration.

The results show that, while MC3T3-E1 cells differentiate into osteoblasts in a BMP-2 (differentiation factor) concentration-dependent manner, ornithine promotes differentiation of osteoblasts to the same level as that by the addition of BMP-2 at higher concentrations, even under low BMP-2 concentration (e.g., BMP-2 concentration 50 ng/mL) conditions.

### Experimental Example 4 Osteoclast Number Measurement Test After Ornithine Addition, by Using Osteoclast Progenitor Cell RAW264

Using the following materials and methods, osteoclast progenitor cells were cultured in ornithine-added or ornithine-free medium. After culture, the number of differentiated osteoclasts was measured, and the osteoclast differentiation suppressive action of ornithine was examined.

### Material

(1) Cell

RAW264 (Riken Cell Bank, RCB0535, lot40)

(2) Medium used

proliferation medium: α-MEM medium, 10% FBS, antibiotic added

test medium: α-MEM medium, 5% FBS, antibiotic added

(3) Test reagent, etc.

α-MEM medium (phenol red free) (Invitrogen, Cat. No. 41061-029)

Penicillin-streptomycin solution (nacalai tesque, Cat. No. 26253-84)

Dulbecco’s PBS (Nissui Pharmaceutical Co., Ltd., Cat. No. 05913)

Cell Count Reagent SF (nacalai tesque, Cat. No. 07553-44)

heparin (SIGMA, Cat. No. H3149-25KU)

RANK ligand, Mouse, Recombinant (SIGMA, Cat. No. R0525-10UG)

TRAP Activity Assay Kit (Immunodaiagnostic Systems, Cat. No. TR103)

L-Ornithine Monohydrochloride (Nacalai, Cat. No. 25718-92)

### Method

### Cell Preculture

The cells were placed in a T-75 flask using a medium and cultured in a CO₂ incubator (5% CO₂, 37° C.). The medium was changed every other day, and the cells were collected when they reached 80% confluence and used for the test.

### Osteoclast Differentiation Test

Cells were prepared in a test medium at 3x10³ cells/0.1 mL/well and seeded in a 96 well plate. The next day, the medium was replaced with a 50 ng/mL RANK ligand (hereinafter to be abbreviated as RANKL) and ornithine 0.02 mg/mL, 0.1 mg/mL, or 0.5 mg/mL addition medium, a non-addition medium, a 100 µg/mL heparin addition medium as positive control, and a RANKL non-addition medium. After culturing for 7 days, TRAP staining was performed, and TRAP staining-positive and multinucleated cells were counted, based on which the ability to suppress differentiation into osteoclasts was examined by comparison. The medium was changed every 3-4 days. The test was performed with n=5.

### Significant Difference Test

The test was a two-sided test with Student’s t-test, and P<0.05 (less than 5% of null hypothesis) or more was judged as significant difference. The data in the graph is shown in mean±standard error.

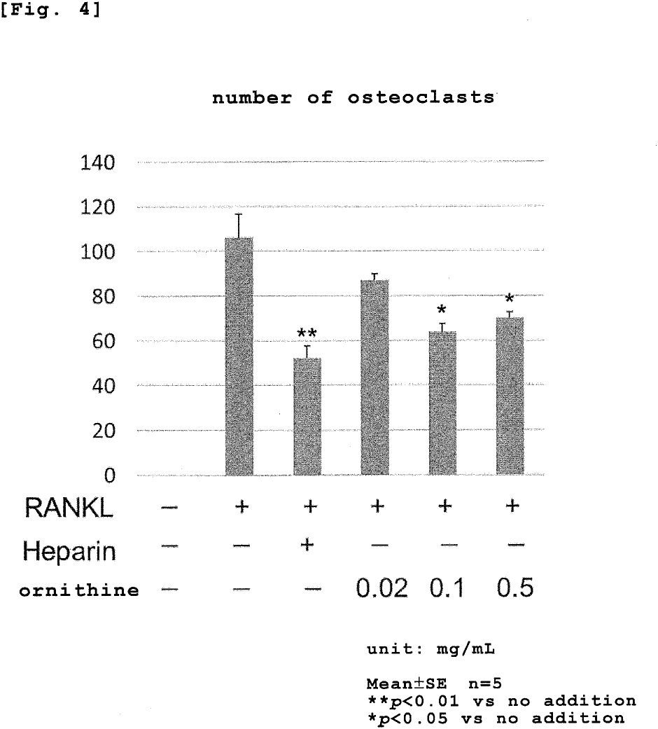

As shown in FIG. 4, as compared with ornithine non-addition (in FIG. 4, the fifth bar graph from the right), the number of osteoclasts significantly decreased by the addition of ornithine 0.1 mg/mL and 0.5 mg/mL, and the number of osteoclasts decreased by the addition of ornithine 0.02 mg/mL.

The results show that ornithine suppresses differentiation of osteoclasts.

### Experimental Example 5: Verification of Bone Density Decrease Suppressive Effect of Ornithine, Using Osteoporosis Mouse Model

To investigate the influence of ornithine on osteoporosis, the bone density decrease suppressive effect of ornithine was evaluated using an osteoporosis model prepared by ovariectomy (OVX) in mice according to the experiment method shown in the following.

### Animal

As the mouse, female, 8-week-old Balb/cA mice purchased from Charles River Laboratories Japan, Inc. were used.
breeding conditions in breeding room:
temperature setting: 23° C.±1° C.
humidity setting: 60%±5%
Lighting time: 07:00-19:00 light period 19:00-07:00 dark period
Feeding: free food ingestion of CRF-1 (ORIENTAL YEAST CO., LTD.)
Water supply: free drinking of tap water

### Preparation of Ornithine Blended Feed

For ingestion of ornithine by mouse, a ornithine blended feed was produced. The composition was based on AIN93G, and was designed such that the total calorie was the same for all compositions by adjusting the amount of corn starch. Each composition is shown in Table 1. The production was performed at room temperature, and all materials were sufficiently mixed with a stirrer and stored in a cool dark place until use.

Each numerical value in Table 1 indicates the blended amount (g) per 1000 g of ornithine blended feed.

TABLE 1

| | ornithine 0% % | ornithine 1% % | ornithine 3% % |
|---|---|---|---|
| vitamin-free casein (ORIENTAL YEAST CO., LTD.) | 200 | 200 | 200 |
| L-cystine (AJINOMOTO CO., INC. ) | 1.8 | 1.8 | 1.8 |
| corn starch | 405.7 | 395.7 | 375.7 |
| α-starch (pregelatinized starch) (ORIENTAL YEAST CO., LTD. ) | 155 | 155 | 155 |
| powdered sugar (sucrose) | 100 | 100 | 100 |
| cellulose (Toyo Roshi Kaisha, Ltd. ) | 50 | 50 | 50 |
| soybean | 40 | 40 | 40 |
| t-butylhydroquinone (FUJIFILM) | 0.008 | 0.008 | 0.008 |
| mineral mix (ORIENTAL YEAST CO., LTD.) | 35 | 35 | 35 |
| AIN93 vitamin mix (ORIENTAL YEAST CO., LTD.) | 10 | 10 | 10 |
| choline hydrogen tartrate (FUJIFILM) | 2.5 | 2.5 | 2.5 |
| L(+)-ornithine monohydrochloride (NACALAI TESQUE, INC.) | 0 | 10 | 30 |
| Total | 1000 | 1000 | 1000 |

amount blended per 1000 g of feed, unit: g

OVX surgical method:
(i) The hair on the back was shaved under isoflurane anesthesia, and an incision of about 8 mm was made around just below the kidney.
(ii) The peritoneum was also incised in the same manner, and the ovary was extracted from the body together with the adipose tissue exposed just below the kidney.
(iii) The upper and lower portions of the ovary were ligated, the ovary was completely removed, and sutured. This was performed on both sides.

The mice that had undergone successful ovariectomy began to gain weight in about one week after the operation, and their body weight increased significantly by the time of autopsy one month later. Therefore, the body weight was monitored after the operation. In addition, since the uterus atrophies significantly after ovariectomy, the success or failure of OVX was determined by measuring the weight of the uterus at autopsy.

In the present specification, the group of mice that underwent ovariectomy (OVX) is referred to as the OVX group. For comparison (control), a group of mice subjected to only shaving, incision of skin and peritoneum, and suturing as sham surgery, without ovariectomy is referred to as Sham group.

### Experiment Method

As shown in Table 2, a comparative test was performed for the Sham (ornithine non-administration) group, the OVX (ornithine non-administration) group, and the OVX (ornithine administration) group.

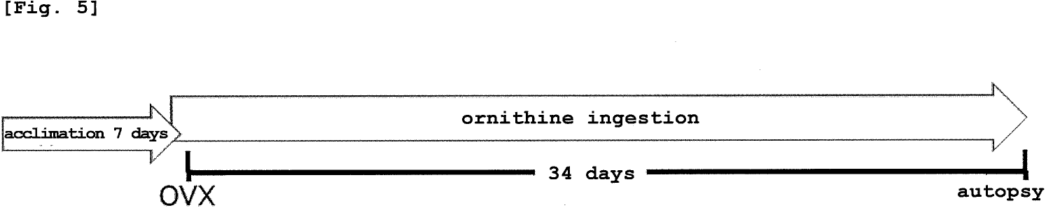

In the OVX (ornithine administration) group, according to the experiment schedule shown in FIG. 5, the diet was switched to 1% ornithine blended feed from 3 days before the OVX surgery, ornithine was provided until immediately before autopsy, and autopsy was performed on the 34 th day after OVX.

In the OVX (ornithine non-administration) group, the experiment was conducted in the same manner as in the OVX (ornithine administration) group, except that an ornithine 0% blended feed was ingested instead of the ornithine 1% blended feed.

In the Sham (ornithine non-administration) group, the experiment was conducted in the same manner as in the OVX (ornithine administration) group, except that the ornithine 0% blended feed was ingested instead of the ornithine 1% blended feed, and sham surgery was performed instead of OVX.

TABLE 2

| group | n | ornithine blended in feed |
|---|---|---|
| Sham (ornithine non-administration) group | 8 | ornithine 0% |
| OVX (ornithine non-administration) group | 8 | ornithine 0% |
| OVX (ornithine administration) group | 8 | ornithine 1% |

### Measurement of Bone Density (BV/TV):

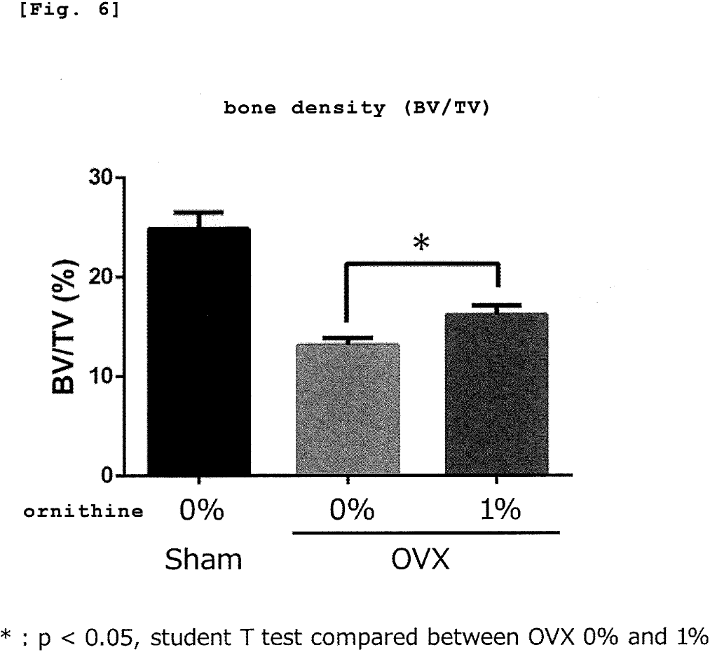

Bone density was measured using femur removed at autopsy. The removed femur was stored at -20° C. until measurement. A micro CT device (Bruker, SKYSCAN) was used to measure bone density. The lower part of the femur was photographed, image was reconstructed (Bruker/NRecon), the bone angle was adjusted, 50 images were cut off from the growth plate reference section, and then 101 images were analyzed (Bruker, DataViewer/CTAn). The area of sponge bone was set and BV/TV was calculated. The results are shown in FIG. 6.

As shown in FIG. 6, in the OVX (ornithine non-administration) group (second bar graph from the left), bone density decreased as compared with the Sham (ornithine non-administration) group (first bar graph from the left). In the OVX (ornithine administration) group (third bar graph from the left), bone density significantly increased as compared with the OVX (ornithine non-administration) group.

From the results, it was confirmed that ornithine significantly suppresses bone density decrease due to OVX. In addition, a bone density decrease suppressive effect in the body by ornithine was confirmed.

### Experimental Example 6: Verification of Muscle Atrophy Suppressive Effect of Ornithine, Using Osteoporosis-Disuse Muscle Atrophy Simultaneous Onset Mouse Model

To verify the effect of ornithine on disuse muscle atrophy that develops simultaneously with osteoporosis, osteoporosis due to OVX and disuse muscle atrophy due to fixation of the lower leg with cast were simultaneously developed in mice according to the experiment method shown below. Using the model, the muscle atrophy suppressive effect of ornithine was evaluated. Production of ornithine blended feed and OVX operation method were the same as those in Experimental Example 5.

### Animal

As the mouse, female, 8-week-old Balb/cA mice purchased from Charles River Laboratories Japan, Inc. were used.
breeding conditions in breeding room:
temperature setting: 23° C.±1° C.
humidity setting: 60%±5%
Lighting time: 07:00-19:00 light period 19:00-07:00 dark period
Feeding: free food ingestion of AIN93G (AJINOMOTO CO., INC., powder)
Water supply: free drinking of tap water

Method of cast fixation of lower leg:
(i) The second joint of the lower right leg of the mouse was bent under isoflurane anesthesia and lightly fixed with a paper adhesive tape or the like.
(ii) The thigh was wrapped around with a 1 cm wide binding tape (TRUSCO, Magic Band (R) Binding Tape (double-sided type)), and fixed by wrapping around the binding tape with another binding tape to completely cover the lower leg.
(iii) Fixation was performed for 9 days, and the tape was removed thereafter.

### Experiment Method

As shown in Table 3, a comparative test was performed for the Sham (ornithine non-administration and no cast fixation) group, the OVX (ornithine non-administration with cast fixation) group, the OVX (ornithine 1% blended feed administration with cast fixation) group, and the OVX (ornithine 3% blended feed administration with cast fixation) group.

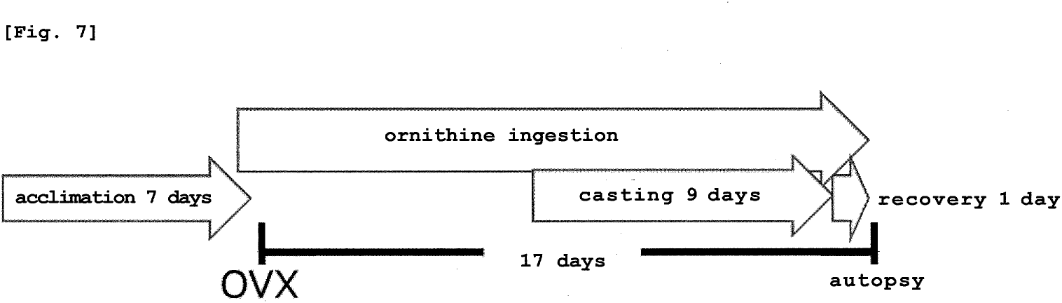

In the OVX (ornithine 1% blended feed administration with cast fixation) group and the OVX (ornithine 3% blended feed administration with cast fixation) group, according to the experiment schedule shown in FIG. 7, the diet was switched to 1% ornithine blended feed or 3% ornithine blended feed from 3 days before the OVX surgery, and ornithine was provided until immediately before autopsy. Cast fixation was applied for 9 days, after which a recovery period of about 24 hr was provided, and then autopsy was performed.

In the OVX (ornithine non-administration with cast fixation) group, the experiment was conducted in the same manner as in the OVX (ornithine 1% blended feed administration with cast fixation) group and the OVX (ornithine 3% blended feed administration with cast fixation) group, except that an ornithine 0% blended feed was ingested instead of the ornithine blended feed.

In the Sham (ornithine non-administration and no cast fixation) group, the experiment was conducted in the same manner as in the OVX (ornithine 1% blended feed administration with cast fixation) group and the OVX (ornithine 3% blended feed administration with cast fixation) group, except that the ornithine 0% blended feed was ingested instead of the ornithine blended feed, sham surgery was performed instead of OVX, and cast fixation was not performed.

TABLE 3

| group | cast fixation | n | ornithine blended in feed |
|---|---|---|---|
| Sham (ornithine non-administration with no cast fixation) group | not fixed | 8 | ornithine 0% |
| OVX (ornithine non-administration with cast fixation) group | fixed | 8 | ornithine 0% |
| OVX (ornithine 1% blended feed administration with cast fixation) group | fixed | 8 | ornithine 1% |
| OVX (ornithine 3% blended feed administration with cast fixation) group | fixed | 8 | ornithine 3% |

### Measurement of Gastrocnemius Muscle Weight

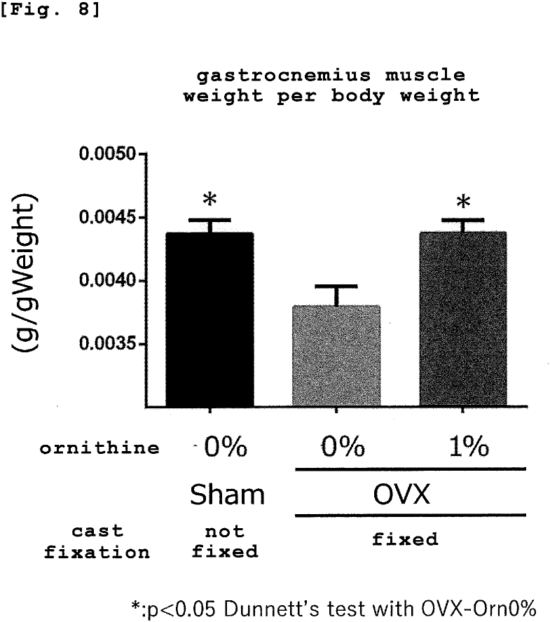

The weight of the gastrocnemius muscle of the Sham (ornithine non-administration with no cast fixation) group, the OVX (ornithine non-administration with cast fixation) group, and the OVX (ornithine 1% blended feed administration with cast fixation) group, which was removed at autopsy, was measured, and the weight per body weight was calculated. The results are shown in FIG. 8.

As shown in FIG. 8, in the OVX (ornithine non-administration with cast fixation) group (second bar graph from the left), gastrocnemius muscle weight decreased as compared with the Sham (ornithine non-administration with no cast fixation) group (first bar graph from the left). In the OVX (ornithine 1% blended feed administration with cast fixation) group (third bar graph from the left), the gastrocnemius muscle weight increased as compared with the OVX (ornithine non-administration with cast fixation) group.

From the results, it was confirmed that ornithine suppresses muscle atrophy simultaneously developed with osteoporosis.

### Experimental Example 7: Verification of Liver Weight Suppressive Effect of Ornithine, Using Osteoporosis Mouse Model

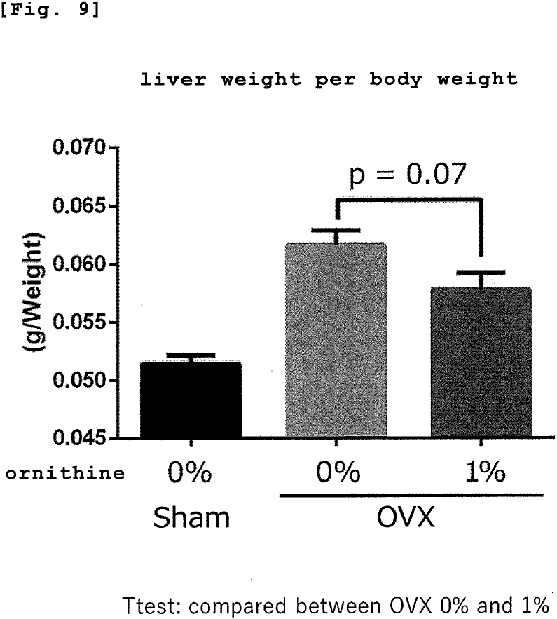

To investigate the influence of ornithine on fatty liver and the like, the weight of the liver of the Sham (ornithine non-administration) group, the OVX (ornithine non-administration) group, and the OVX (ornithine administration) group in Experimental Example 5, which was removed at autopsy, was measured, and the liver weight per body weight was compared. The results are shown in FIG. 9.

As shown in FIG. 9, in the OVX (ornithine non-administration) group (second bar graph from the left), liver weight per body weight increased as compared with the Sham (ornithine non-administration) group (first bar graph from the left). In the OVX (ornithine administration) group (third bar graph from the left), liver weight decreased as compared with the OVX (ornithine non-administration) group.

The results show that ornithine suppresses liver weight increase which is an index of fatty liver induced by OVX.

### Experimental Example 8: Gene Expression Analysis

To investigate the influence of ornithine on lifestyle-related disease-related factors, the gene expression analysis of the femur and gastrocnemius muscle removed at autopsy in Experimental Example 6, and the liver removed at autopsy in Experimental Example 5 was performed.

Muscle and the like were detached from the femur after removal, and the femur was immediately frozen in liquid nitrogen. The gastrocnemius muscle and the liver were also similarly frozen in liquid nitrogen immediately after removal. Using Multi-beads Shocker (YASUI KIKAI), the samples were pulverized, and RNA was extracted by a conventional method (NIPPON GENE CO., LTD./ISOGEN II). Using the extracted RNA, reverse transcription was performed, and cDNA was acquired (Takara Bio Inc./PrimeScript 1^{st}strand cDNA Synthesis Kit). Using the cDNA, the expression levels of osteocalcin (osteoblast marker) and cyclophilin A (housekeeping gene) were analyzed for the femur (Thermo Fisher SCIENTIFIC/QuantStudio 3, TaqMan Fast Advanced Master Mix). As for the gastrocnemius muscle, muscle atrophy markers MuRF1 and Atg3, and 18S (housekeeping gene) were measured. For the liver, SREBP1c, MTP, and PPARγ, which are the indices of lifestyle-related diseases, and cyclophilin A (housekeeping gene) were measured. The list of primers is shown in Table 4.

TABLE 4

Real-time primer base sequence

| | | |
|---|---|---|
| Gene | Forward primer | Reverse primer |
| Osteocalcin | ACGGTATCACTATTTAGGACCTGTG (SEQ ID NO:1) | ACTTTATTTTGGAGCTGCTGTGAC (SEQ ID NO:2) |
| Cyclophilin A | TGGAGAGCACCAAGACAGACA (SEQ ID NO:3) | TGCCGGAGTCGACAATGAT (SEQ ID NO:4) |
| MuRF1 | GCTGGTGGAAAACATCATTGACA (SEQ ID NO: 5) | CATCGGGGTGGCTGCCTTT (SEQ ID NO: 6) |
| Atg3 | CTGGAGATCACTTAGTCCACCA (SEQ ID NO:7) | GTCGGAAGATATGCCTTCACTTT (SEQ ID NO:8) |
| 18S | AACGCCACTTGTCCCTCTAA (SEQ ID NO: 9) | GTGGAGCGATTTGTCTGGTT (SEQ ID NO:10) |
| SREBPIc | GCCATGGATTGCACATTTG (SEQ ID NO:11) | TCAGGAGAGTTGGCACCTG (SEQ ID NO:12) |
| MTP | CCAGCATGATCCTCTTGGCA (SEQ ID NO:13) | TGAGAGGCCAGTTGTGTGAC (SEQ ID NO:14) |
| PPARγ | CGCTGATGCACTGCCTATGA (SEQ ID NO:15) | CGAGTGGTCTTCcATCACGG (SEQ ID NO:16) |

### Statistical Analysis

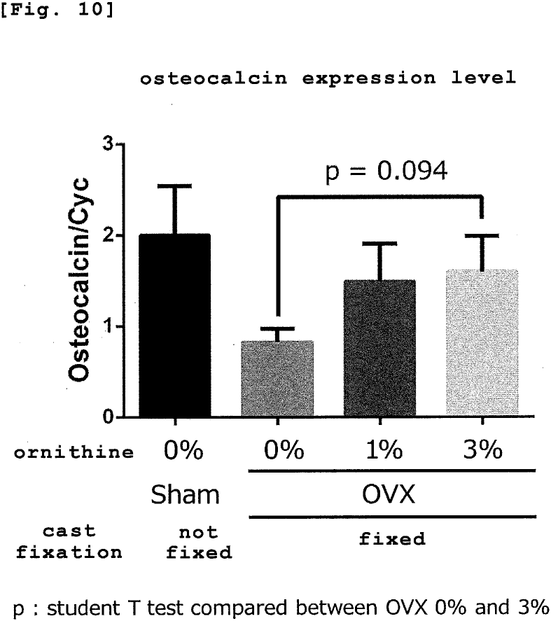

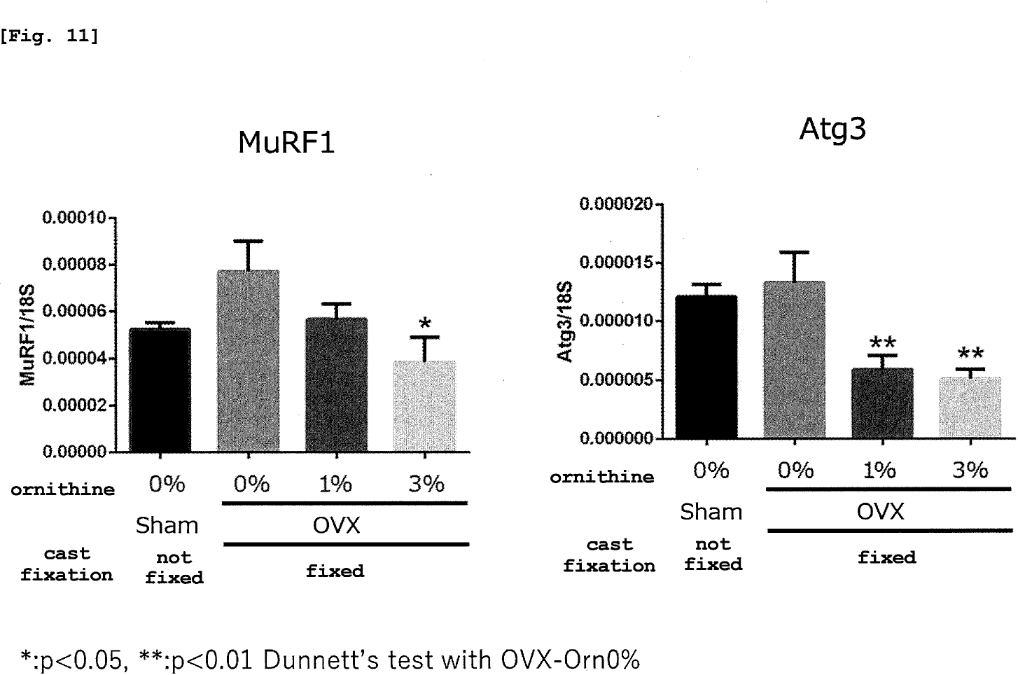

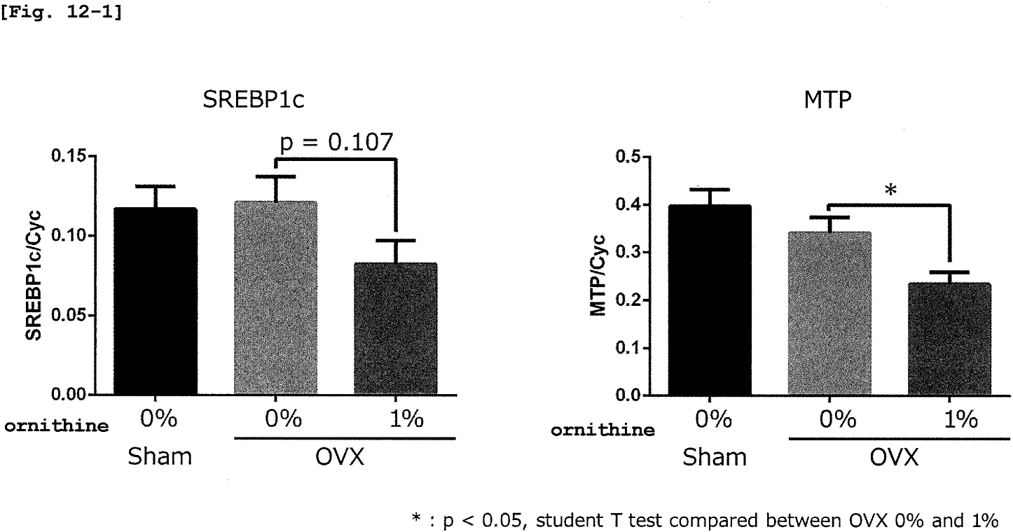

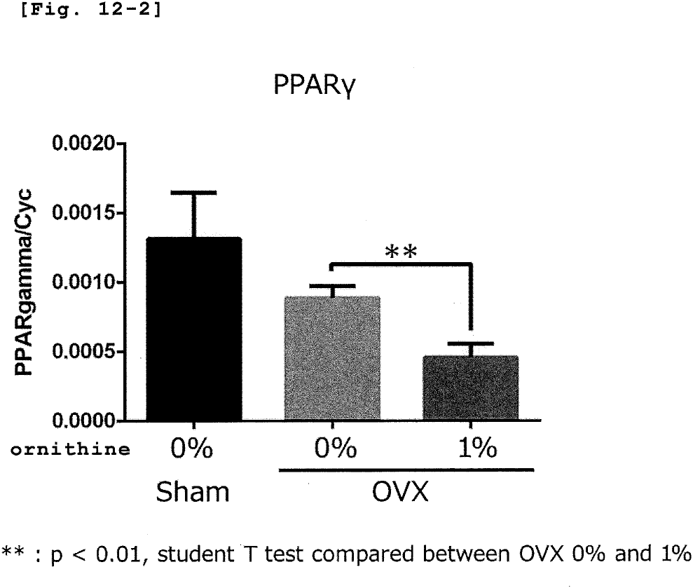

The data was shown graphically using mean and SE values, and Student’s t test was used for comparison of two groups and Dunnett test was used for comparison of three or more groups. For the analysis, Prism6 for Windows (GraphPad) was used. The results are shown in FIG. 10, FIG. 11, FIGS. 12-1, and FIGS. 12-2.

As shown in FIG. 10, in the OVX (ornithine non-administration with cast fixation) group (second bar graph from the left), osteocalcin expression level decreased as compared with the Sham (ornithine non-administration with no cast fixation) group (first bar graph from the left). In the OVX (ornithine 1% blended feed administration with cast fixation) group (third bar graph from the left) and the OVX (ornithine 3% blended feed administration with cast fixation) group (fourth bar graph from the left), the osteocalcin expression level increased as compared with the OVX (ornithine non-administration with cast fixation) group.

The results show that ornithine has an osteocalcin expression inducing action in vivo as well.

As shown in the left figure and the right figure in FIG. 11, in the OVX (ornithine non-administration with cast fixation) group (second bar graph from the left), the expression levels of MuRF1 and Atg3 increased as compared with the Sham (ornithine non-administration with no cast fixation) group (first bar graph from the left). However, in the OVX (ornithine 1% blended feed administration with cast fixation) group (third bar graph from the left) and the OVX (ornithine 3% blended feed administration with cast fixation) group (fourth bar graph from the left), the expression levels of MuRF1 and Atg3 decreased as compared with the OVX (ornithine non-administration with cast fixation) group.

The results show that ornithine has an action of suppressing the expression of MuRF1 and Atg3.

As shown in the left figure and the right figure in FIGS. 12-1, and FIGS. 12-2, in the OVX (ornithine administration) group (third bar graph from the left), the expression levels of SREBP1c, MTP, and PPARγdecreased as compared with the OVX (ornithine non-administration) group (second bar graph from the left).

The results show that ornithine has an action of suppressing the expression of SREBP1c, MTP, and PPARγin the liver.

### Industrial Applicability

According to the present invention, a composition effective for preventing or improving bone strength decrease and the like can be provided.

We claim:

1. A composition for preventing or improving bone strength decrease, comprising ornithine as an active ingredient.

2. The composition according to claim 1, wherein the bone strength decrease is caused by menopause, immobilization osteoporosis, disuse osteoporosis, and/or aging.

3. The composition according to claim 1, wherein the bone strength decrease is caused by bone density decrease or bone quality decrease.

4. The composition according to claim 3, wherein the bone quality decrease is caused by bone metabolism decrease.

5. The composition according to claim 1, wherein the composition comprises an osteoblast differentiation promoter and/or an osteoclast differentiation inhibitor.

6. The composition according to claim 1, wherein the composition is able to prevent or improve musculoskeletal function decrease.

7. The composition according to claim 1, wherein the composition is formulated for ingestion by a subject with a decreased BMP-2 signal.

8. The composition according to claim 7, wherein the subject with a decreased BMP-2 signal is a postmenopausal woman, an osteoporosis patient, or an elderly person.

9. A composition for inducing osteocalcin expression, comprising ornithine as an active ingredient.

10. A composition for preventing or improving changes in physical condition due to changes in postmenopausal hormone imbalance, comprising ornithine as an active ingredient.

11. The composition according to claim 10, wherein the change in the physical condition is caused by a bone strength decrease and/or a liver weight increase.

12. The composition according to claim 1, wherein the composition is a food.

13. The composition according to claim 9, wherein said composition is indicated for a function resulting from osteocalcin expression induction.

14. The composition according to claim 13, wherein the function is selected from the group consisting of “prevention of osteoporosis”, “strengthening bone”, “promotion of regeneration of bone”, “bone building capacity”, “suppression of bone fracture”, and “acceleration of recovery from bone fracture”.

15. A composition for suppressing the expression of MuRF1 and/or Atg3, comprising ornithine as an active ingredient.

16. The composition according to claim 15, wherein said composition is indicated for a function resulting from suppressing the expression of MuRF1 and/or Atg3.

17. The composition according to claim 16, wherein the function is selected from the group consisting of “maintaining body in movable state for a long time”, “maintaining walking function”, “maintaining exercise function”, and “being able to walk for a long time”.

18. A composition for suppressing the expression of SREBP1c and/or MTP and/or PPARy, comprising ornithine as an active ingredient.

19. The composition according to claim 18, wherein the composition is indicated for a function resulting from suppressing the expression of SREBP1c and/or MTP and/or PPARy.

20. A method for preventing or improving a bone strength decrease in a subject in need of the prevention or improvement of bone strength decrease, comprising administering a composition comprising an effective amount of ornithine to the subject.

21. The method according to claim 20, wherein the bone strength decrease is caused by menopause, immobilization osteoporosis, disuse osteoporosis, and/or aging.

22. The method according to claim 20, wherein the bone strength decrease is caused by bone density decrease or bone quality decrease.

23. The method according to claim 22, wherein the bone quality decrease is caused by bone metabolism decrease.

24. The method according to claim 20, wherein the prevention or improvement of bone strength decrease is achieved by the promotion of osteoblast differentiation and/or suppression of osteoclast differentiation.

25. The method according to claim 20, wherein the subject is in further need of the prevention or improvement of musculoskeletal function decrease.

26. The method according to claim 20, wherein the subject has decreased BMP-2 signal.

27. The method according to claim 26, wherein the subject with a decreased BMP-2 signal is a postmenopausal woman, an osteoporosis patient, or an elderly person.

28. A method for inducing osteocalcin expression in a subject in need of the induction of osteocalcin expression, comprising administering a composition comprising an effective amount of ornithine to the subject.

29. A method for preventing or improving changes in physical condition due to changes in postmenopausal hormone balance in a subject in need of the prevention or improvement of changes in physical condition due to changes in postmenopausal hormone balance, comprising administering a composition comprising an effective amount of ornithine to the subject.

30. The method according to claim 29, wherein the change in the physical condition is caused by bone strength decrease and/or liver weight increase.

31. The method according to claim 20, wherein the composition is orally administered.

32. A method for suppressing the expression of MuRF1 and/or Atg3 in a subject in need of the suppression of the expression of MuRF1 and/or Atg3, comprising administering a composition comprising an effective amount of ornithine to the subject.

33. A method for suppressing the expression of SREBP1c and/or MTP and/or PPARy in a subject in need of the suppression of the expression of SREBP1c and/or MTP and/or PPARy, comprising administering a composition comprising an effective amount of ornithine to the subject.


SEQUENCE LISTING (16 sequences)

SEQ ID NO: 1 - DNA, 25 bp, synthetic construct
  misc_feature 1..25: note = primer
  source 1..25: mol_type = other DNA
acggtatcactatttaggacctgtg

SEQ ID NO: 2 - DNA, 24 bp, synthetic construct
  misc_feature 1..24: note = primer
  source 1..24: mol_type = other DNA
actttattttggagctgctgtgac

SEQ ID NO: 3 - DNA, 21 bp, synthetic construct
  misc_feature 1..21: note = primer
  source 1..21: mol_type = other DNA
tggagagcaccaagacagaca

SEQ ID NO: 4 - DNA, 19 bp, synthetic construct
  misc_feature 1..19: note = primer
  source 1..19: mol_type = other DNA
tgccggagtcgacaatgat

SEQ ID NO: 5 - DNA, 23 bp, synthetic construct
  misc_feature 1..23: note = primer
  source 1..23: mol_type = other DNA
gctggtggaaaacatcattgaca

SEQ ID NO: 6 - DNA, 19 bp, synthetic construct
  misc_feature 1..19: note = primer
  source 1..19: mol_type = other DNA
catcggggtggctgccttt

SEQ ID NO: 7 - DNA, 22 bp, synthetic construct
  misc_feature 1..22: note = primer
  source 1..22: mol_type = other DNA
ctggagatcacttagtccacca

SEQ ID NO: 8 - DNA, 23 bp, synthetic construct
  misc_feature 1..23: note = primer
  source 1..23: mol_type = other DNA
gtcggaagatatgccttcacttt

SEQ ID NO: 9 - DNA, 20 bp, synthetic construct
  misc_feature 1..20: note = primer
  source 1..20: mol_type = other DNA
aacgccacttgtccctctaa

SEQ ID NO: 10 - DNA, 20 bp, synthetic construct
  misc_feature 1..20: note = primer
  source 1..20: mol_type = other DNA
gtggagcgatttgtctggtt

SEQ ID NO: 11 - DNA, 19 bp, synthetic construct
  misc_feature 1..19: note = primer
  source 1..19: mol_type = other DNA
gccatggattgcacatttg

SEQ ID NO: 12 - DNA, 19 bp, synthetic construct
  misc_feature 1..19: note = primer
  source 1..19: mol_type = other DNA
tcaggagagttggcacctg

SEQ ID NO: 13 - DNA, 20 bp, synthetic construct
  misc_feature 1..20: note = primer
  source 1..20: mol_type = other DNA
ccagcatgatcctcttggca

SEQ ID NO: 14 - DNA, 20 bp, synthetic construct
  misc_feature 1..20: note = primer
  source 1..20: mol_type = other DNA
tgagaggccagttgtgtgac

SEQ ID NO: 15 - DNA, 20 bp, synthetic construct
  misc_feature 1..20: note = primer
  source 1..20: mol_type = other DNA
cgctgatgcactgcctatga

SEQ ID NO: 16 - DNA, 20 bp, synthetic construct
  misc_feature 1..20: note = primer
  source 1..20: mol_type = other DNA
cgagtggtcttccatcacgg